SEQUENCE LISTING (17 sequences)

SEQ ID NO: 1 - RNA, 22 nt, artificial sequence
  synthetic PNA
acuccgcguggccucggucgug

SEQ ID NO: 2 - RNA, 26 nt, artificial sequence
  synthetic PNA
aagaagcuauuaaaaucacaugggga

SEQ ID NO: 3 - RNA, 23 nt, artificial sequence
  synthetic PNA
uaggcagcucucccuagcauugu

SEQ ID NO: 4 - RNA, 22 nt, artificial sequence
  synthetic 2′-FANA oligonucleotide
acuccgcguggccucggucgug

SEQ ID NO: 5 - RNA, 26 nt, artificial sequence
  synthetic 2′-FANA oligonucleotide
aagaagcuauuaaaaucacaugggga

SEQ ID NO: 6 - RNA, 23 nt, artificial sequence
  synthetic 2′-FANA oligonucleotide
uaggcagcucucccuagcauugu

SEQ ID NO: 7 - RNA, 22 nt, artificial sequence
  synthetic LNA oligonucleotide
acuccgcguggccucggucgug

SEQ ID NO: 8 - RNA, 26 nt, artificial sequence
  synthetic LNA oligonucleotide
aagaagcuauuaaaaucacaugggga

SEQ ID NO: 9 - RNA, 23 nt, artificial sequence
  synthetic LNA oligonucleotide
uaggcagcucucccuagcauugu

SEQ ID NO: 10 - RNA, 41 nt, artificial sequence
  SARS-CoV s2m
uucaucgaggccacgcggaguacgaucgaggguacagugaa

SEQ ID NO: 11 - RNA, 41 nt, artificial sequence
  SARS-CoV-2 s2m
uucaccgaggccacgcggaguacgaucgaguguacagugaa

SEQ ID NO: 12 - RNA, 22 nt, artificial sequence
  miR-1307-3p
acucggcguggcgucggucgug

SEQ ID NO: 13 - RNA, 18 nt, artificial sequence
  SARS-CoV-2  3′ UTR 100 - 117
cuuuaaucucacauagca

SEQ ID NO: 14 - RNA, 42 nt, artificial sequence
  SARS-CoV-2  3′ UTR Terminus
ugcuauccccaugugauuuuaauagcuucuuaggagaaugac

SEQ ID NO: 15 - RNA, 20 nt, artificial sequence
  miR-760-3p
cggcucugggucugugggga

SEQ ID NO: 16 - RNA, 67 nt, artificial sequence
  SARS-CoV-2 DIS-s2m Extended
cauuuucaccgaggccacgcggaguacgaucgaguguacagugaacaaugcuagggagagcugccua

SEQ ID NO: 17 - RNA, 22 nt, artificial sequence
  miR-34a-5p
uggcagugucuuagcugguugu

(19) United States
(12) Patent Application Publication
(10) Pub. No.: US 2022/0119812 A1
(43) Pub. Date: Apr. 21, 2022

(54) MICRO RNA INTERACTIONS AS THERAPEUTIC TARGETS FOR COVID-19 AND OTHER VIRAL INFECTIONS

(71) Applicant: DUQUESNE UNIVERSITY OF THE HOLY SPIRIT, Pittsburgh, PA (US)

(72) Inventors: Mihaela Rita Mihailescu, Pittsburgh, PA (US); Jeffrey D. Evanseck, Pittsburgh, PA (US); Joshua A. Imperatore, Canonsburg, PA (US); Kendy Anne Marie Guarinoni, Pittsburgh, PA (US); Caylee Lyne Cunningham, East Liverpool, OH (US); Caleb James Frye, Manor, PA (US); Adam Henry Kensinger, Pittsburgh, PA (US)

(21) Appl. No.: 17/451,521

(22) Filed: Oct. 20, 2021

Related U.S. Application Data
(60) Provisional application No. 63/094,036, filed on Oct. 20, 2020.

(51) Int. Cl. C12N 15/113; A61P 31/14

(52) U.S. Cl. CPC C12N 15/113; C12N 2310/141; C12N 2310/3231; A61P 31/14

(57) ABSTRACT

Provided is an agent that binds to a SARS-CoV-2 s2m motif, a SARS-CoV-2 3′-UTR Terminus, or a SARS-CoV-2 DIS-s2m extended sequence. Provided is a method of treating an infection in a subject, comprising: administering a therapeutically effective amount of the agent to the subject. In some embodiments, the infection is SARS-CoV-2 infection.

### CROSS-REFERENCE TO RELATED APPLICATIONS

This invention claims the benefit of U.S. Provisional Application No. 63/094,036 filed on Oct. 20, 2020, which is hereby incorporated by reference in its entirety.

### GOVERNMENT SUPPORT

This invention was made with government support under grant CHE-2029124 awarded by the National Science Foundation (NSF). The government has certain rights in the invention.

### SEQUENCE LISTING

The instant application contains a Sequence Listing which is submitted in ASCII format via EFS-Web and is hereby incorporated by reference in its entirety. The ASCII copy, created on Oct. 20, 2021, is named 049459-00491_SequenceListing.txt and is 3,878 bytes in size.

### FIELD

The invention relates to the s2m motif as a novel therapeutic target in the SARS-CoV-2 virus and other related viruses, as well as methods for precluding, mitigating, or treating infection with SARS-CoV-2 and other related viruses. Additionally, the invention relates to the interactions between the s2m motif (nucleotides (nt) 29,727-29,768, Global Initiative on Sharing Avian Influenza Data (GISAID) Accession Number EPI_ISL_402123) and the host miR-1307-3p, between the SARS-CoV-2 3′-untranslated region terminal 42-(nt 29,828-29,870 GISAID Accession Number EPI_ISL_402123) and the host miR-760-3p, and between an extension of the s2m motif (nt 29,768-29,790, GISAID Accession Number EPI_ISL_402123) and miR-34a-5p as novel therapeutic targets in the SARS-CoV-2 virus and other viruses, as well as methods for precluding, mitigating, or treating SARS-CoV-2 and other related viruses.

### BACKGROUND

The severe acute respiratory syndrome coronavirus 2 (SARS-CoV-2) was first detected in Wuhan, Hubei Province, People's Republic of China in December of 2019, causing outbreaks of COVID-19 that as of Sep. 20, 2021, infected 219 million people with approximately 4.55 million deaths.

SARS-CoV-2, the virus causing the COVID-19 pandemic, belongs to the Nidovirales order, the Coronaviridae family, Betacoronavirus genus, lineage B. The viruses of the Nidovirales order are enveloped, positive-stranded RNA(+) viruses, with very large genomes (˜30 kb for coronaviruses), all of which share a similar organization. Starting at the 5′ end, the RNA genome encodes the replicase, which has two open reading frames (ORF1a and ORF1b), followed by the structural proteins, spike (S), envelope (E), membrane (M) and nucleocapsid (N) proteins. The N proteins interact with the genomic RNA to form the nucleocapsid, which is enveloped by a membrane decorated on the surface with S, E and M proteins.

The COVID-19 pandemic highlights the lack of detailed knowledge about the life cycle of coronaviruses in general and about their interactions with the host, as well as the absence of effective antivirals for treatment, pointing towards the lack of preparedness to deal with the current and future pandemics.

The 5′- and 3′-untranslated regions (UTR) of the SARS-CoV-2 genomic RNA(+) contain conserved structures that are important for its replication. The 3′-UTR contains a bulged stem-loop (BSL), the region LI connecting it with a pseudoknot (PK) stem-loop, followed by a hypervariable region (HVR). Although most of the 3′-UTR HVR sequence is not conserved, it harbors a 41-nucleotide sequence named the s2m motif which is highly conserved not only in the Coronaviridae family, but also in three other families of RNA(+) viruses, the Astroviridae, Caliciviridae and Picornaviridae. The s2m motif confers a selective advantage; however, its exact function is unknown. The presence of the s2m motif in multiple viral families indicates a role that might be independent of the steps of a particular viral life cycle, possibly being involved in interactions with the host. Furthermore, the viral UTRs show potential for RNA-RNA interactions, such as those with host microRNAs (miRNAs). Screening assays of SARS-CoV infection indicate a downregulation of select miRNAs; while the mechanism of this downregulation is not known, predicted viral-host RNA-RNA interactions could be a mechanism of viral hijacking. Conservation of these binding sites through SARS-CoV and SARS-CoV-2 supports a potential beneficial role in viral proliferation.

There is a need to determine the life cycle of the SARS-CoV-2 virus, as well as to characterize the virus interactions with the host, in order to develop antivirals and treatment methods to preclude and/or treat the onset and progression of this virus.

### SUMMARY OF THE INVENTION

The invention provides novel therapeutic targets for SARS-CoV-2: (i) in the form of an s2m motif (nt 29,727-29,768, Global Initiative on Sharing Avian Influenza Data (GISAID) Accession Number EPI_ISL_402123) that interacts and binds with a cellular microRNA, miR-1307-3p, (ii) in the form of a sequence located in the last 42-nucleotides of the genome 3′-UTR (nt 29,828-29,870 GISAID Accession Number EPI_ISL_402123) henceforth named 3′-UTR Terminus) that interacts and binds with a cellular microRNA, miR-760-3p, and (iii) in the form of a sequence located at positions nt 29,768-29,790 (GISAID Accession Number EPI_ISL_402123) of the genome downstream of the 3′-UTR s2m motif (henceforth named dimer initiation site s2m extended, or DIS-s2m extended) that interacts and binds with a cellular microRNA, miR-34a-5p.

In certain embodiments, the s2m motif binds two or more molecules of the cellular microRNA, miR-1307-3p, the viral 3′-UTR Terminus binds one molecule of the cellular microRNA miR-760-3p and DIS-s2m extended motif binds one molecule of the microRNA miR-34a-5p.

Provided is an agent that binds to a SARS-CoV-2 s2m motif, a SARS-CoV-2 3′-UTR Terminus, or a SARS-CoV-2 DIS-s2m extended sequence.

In some embodiments, binding of the agent to the SARS-CoV-2 s2m prevents and/or disrupts interaction of the s2m motif with miR-1307-3p.

In some embodiments, binding of the agent to the SARS-CoV-2 3′-UTR Terminus prevents and/or disrupts interaction of the 3′-UTR Terminus with miR-760-3p.

In some embodiments, binding of the agent to the SARS-CoV-2 DIS-s2m extended sequence prevents and/or disrupts interaction of the DIS-s2m extended sequence with miR-34a-5p

In some embodiments, the agent comprises an engineered peptide nucleic acid (PNA). In further embodiments, the PNA is a gamma PNA.

In some embodiments, the PNA or gamma PNA comprises a sequence selected from the group consisting of:

(SEQ ID NO: 1; PNA-1307)
ACUCCGCGUGGCCUCGGUCGUG;
(SEQ ID NO: 2; PNA-760)
AAGAAGCUAUUAAAAUCACAUGGGGA;
and
(SEQ ID NO: 3; PNA-34a)
UAGGCAGCUCUCCCUAGCAUUGU.

In some embodiments, the PNA or gamma PNA further comprises a C-terminal lysine residue and is selected from the group consisting of:

PNA-1307
(SEQ ID NO: 1)
[H-ACUCCGCGUGGCCUCGGUCGUG-Lys-NH₂];
PNA-760
(SEQ ID NO: 2)
[H-AAGAAGCUAUUAAAAUCACAUGGGGA-Lys-NH₂];
and
PNA-34a
(SEQ ID NO: 3)
[H-UAGGCAGCUCUCCCUAGCAUUGU-Lys-NH₂].

In further embodiments, the PNA or gamma PNA comprises a sequence selected from the group consisting of SEQ ID NO: 1 or a sequence having about 50%, 60%, 70%, 80%, 90%, 91%, 92%, 93%, 94%, 95%, 96%, 97%, 98%, or 99% sequence identity thereto, optionally comprising a C-terminal lysine residue. In further embodiments, the PNA or gamma PNA comprises a sequence selected from the group consisting of SEQ ID NO: 2 or a sequence having about 50%, 60%, 70%, 80%, 90%, 91%, 92%, 93%, 94%, 95%, 96%, 97%, 98%, or 99% sequence identity thereto, optionally comprising a C-terminal lysine residue. In further embodiments, the PNA or gamma PNA comprises a sequence selected from the group consisting of SEQ ID NO: 3 or a sequence having about 50%, 60%, 70%, 80%, 90%, 91%, 92%, 93%, 94%, 95%, 96%, 97%, 98%, or 99% sequence identity thereto, optionally comprising a C-terminal lysine residue.

In some embodiments the agent comprises 2′-deoxy 2′-fluoroarabino (2′-FANA) oligonucleotides. In further embodiments the 2′-FANA oligonucleotides comprise a sequence selected from the group consisting of:

(SEQ ID NO: 4; FANA-1307)
ACUCCGCGUGGCCUCGGUCGUG;
(SEQ ID NO: 5; FANA-760)
AAGAAGCUAUUAAAAUCACAUGGGGA;
and
(SEQ ID NO: 6; FANA-34a)
UAGGCAGCUCUCCCUAGCAUUGU.

In further embodiments, the 2′-FANA comprises a sequence selected from the group consisting of SEQ ID NO: 4 or a sequence having about 50%, 60%, 70%, 80%, 90%, 91%, 92%, 93%, 94%, 95%, 96%, 97%, 98%, or 99% sequence identity thereto. In yet further embodiments, the 2′-FANA comprises a sequence selected from the group consisting of SEQ ID NO: 4 or a sequence having at least 50%, 60%, 70%, 80%, 90%, 91%, 92%, 93%, 94%, 95%, 96%, 97%, 98%, or 99% sequence identity thereto. In further embodiments, the 2′-FANA comprises a sequence selected from the group consisting of SEQ ID NO: 5 or a sequence having about 50%, 60%, 70%, 80%, 90%, 91%, 92%, 93%, 94%, 95%, 96%, 97%, 98%, or 99% sequence identity thereto. In yet further embodiments, the 2′-FANA comprises a sequence selected from the group consisting of SEQ ID NO: 5 or a sequence having at least 50%, 60%, 70%, 80%, 90%, 91%, 92%, 93%, 94%, 95%, 96%, 97%, 98%, or 99% sequence identity thereto. In further embodiments, the 2′-FANA comprises a sequence selected from the group consisting of SEQ ID NO: 6 or a sequence having about 50%, 60%, 70%, 80%, 90%, 91%, 92%, 93%, 94%, 95%, 96%, 97%, 98%, or 99% sequence identity thereto. In yet further embodiments, the 2′-FANA comprises a sequence selected from the group consisting of SEQ ID NO: 6 or a sequence having at least 50%, 60%, 70%, 80%, 90%, 91%, 92%, 93%, 94%, 95%, 96%, 97%, 98%, or 99% sequence identity thereto.

In some embodiments, the agent comprises locked nucleic acid (LNA) oligonucleotides. In further embodiments, the LNA oligonucleotides comprise a sequence selected from the group consisting of:

(SEQ ID NO: 7; LNA-1307)
ACUCCGCGUGGCCUCGGUCGUG;
(SEQ ID NO: 8; LNA-760)
AAGAAGCUAUUAAAAUCACAUGGGGA;
and
(SEQ ID NO: 9; LNA-34a)
UAGGCAGCUCUCCCUAGCAUUGU.

In further embodiments, the LNA comprises a sequence selected from the group consisting of SEQ ID NO: 7 or a sequence having about 50%, 60%, 70%, 80%, 90%, 91%, 92%, 93%, 94%, 95%, 96%, 97%, 98%, or 99% sequence identity thereto. In yet further embodiments, the LNA comprises a sequence selected from the group consisting of SEQ ID NO: 7 or a sequence having at least 50%, 60%, 70%, 80%, 90%, 91%, 92%, 93%, 94%, 95%, 96%, 97%, 98%, or 99% sequence identity thereto. In further embodiments, the LNA comprises a sequence selected from the group consisting of SEQ ID NO: 8 or a sequence having about 50%, 60%, 70%, 80%, 90%, 91%, 92%, 93%, 94%, 95%, 96%, 97%, 98%, or 99% sequence identity thereto. In yet further embodiments, the LNA comprises a sequence selected from the group consisting of SEQ ID NO: 8 or a sequence having at least 50%, 60%, 70%, 80%, 90%, 91%, 92%, 93%, 94%, 95%, 96%, 97%, 98%, or 99% sequence identity thereto. In further embodiments, the LNA comprises a sequence selected from the group consisting of SEQ ID NO: 9 or a sequence having about 50%, 60%, 70%, 80%, 90%, 91%, 92%, 93%, 94%, 95%, 96%, 97%, 98%, or 99% sequence identity thereto. In yet further embodiments, the LNA comprises a sequence selected from the group consisting of SEQ ID NO: 9 or a sequence having at least 50%, 60%, 70%, 80%, 90%, 91%, 92%, 93%, 94%, 95%, 96%, 97%, 98%, or 99% sequence identity thereto.

In some embodiments, the agent comprises a small molecule.

Provided is a method of treating an infection in a subject, comprising: administering a therapeutically effective amount of the agent of any one of the preceding embodiments to the subject. In some embodiments, the infection is SARS-CoV-2 infection.

Also provided is a composition comprising the agent.

Provided is a method of treating an infection in a subject, comprising: administering a therapeutically effective amount of the composition to the subject. In some embodiments, the infection is SARS-CoV-2 infection.

Provided is a method of treating or preventing the onset of a cytokine storm in a SARS-CoV-2 subject, comprising: administering a therapeutically effective amount of the agent or of the composition to the subject.

The invention also provides a method for treating SARS-CoV-2 infection. The method includes targeting a SARS-CoV-2 s2m motif directly and/or its dimerization; introducing small molecules or antisense molecules to the s2m motif; and preventing and/or disrupting interactions of the s2m motif with miR-1307-3p, thereby releasing the miR-1307-3p to perform its normal cellular functions. The method also includes targeting the SARS-CoV-2 3′-UTR Terminus interactions with miR-760-3p; and preventing and/or disrupting these interactions, thereby releasing the miR-760-3p to perform its normal cellular functions. Additionally, the method includes targeting SARS-CoV-2 DIS-s2m extended interactions with miR-34a-5p; and preventing and/or disrupting interactions of the DiS-s2m extended with miR-34a-5p, thereby releasing the miR-34a-5p to perform its normal cellular functions.

The invention further provides a method for preventing the onset of a cytokine storm in a SARS-CoV-2 subject. The method includes targeting a SARS-CoV-2 s2m motif directly and/or its dimerization; introducing small molecules or antisense molecules to the s2m motif; and preventing and/or disrupting interactions and binding of the s2m motif with miR-1307-3p, thereby releasing the miR-1307-3p to perform its normal cellular function to prevent the onset of the cytokine storm. The method also includes targeting a SARS-CoV-2 3′-UTR Terminus introducing small molecules or antisense molecules that bind the sequence; and preventing and/or disrupting interactions and binding of the 3′-UTR sequence with miR-760-3p, thereby releasing the miR-760-3p to perform its normal cellular function to prevent the onset of the cytokine storm. Additionally, the method includes targeting SARS-CoV-2 DiS-s2m extended directly by introducing small molecules or antisense molecules to the binding site of miR-34a-5p, thereby releasing miR-34a-5p to perform its normal cellular function.

In certain embodiments, the small molecules or antisense molecules are selected from the group consisting of 2′-fluoro-2′-deoxyarabinonucleic acids (FANAs), peptide nucleic acids (PNAs), locked nucleic acids (LNAs), and combinations thereof.

In addition, the invention provides an anti-viral therapy to treat or mitigate SARS-CoV-2 in a subject that includes a therapeutic target in a form of an s2m motif, in a form of a 3′-UTR Terminus sequence, and in the form of a DIS-s2m extended sequence; and small molecules or antisense molecules to prevent and/or disrupt interactions and binding of the s2m motif with miR-1307-3p, the 3′-UTR Terminus with miR-760-3p, and the DIS-s2m extended with miR-34a-5p.

Moreover, the invention provides a method of treating or mitigating SARS-CoV-2 in a subject. The method includes identifying a subject having SARS-CoV-2; and administering a therapeutically effective amount of an anti-viral compound to treat or mitigate the virus.

### BRIEF DESCRIPTION OF THE DRAWINGS

FIG. 1 is a schematic drawing of the predicted SARS-CoV-2 s2m motif, created using RNAStructure and Structure Editor software packages. Nucleotide variances compared to SARS-CoV are highlighted in black.

FIG. 2 illustrates SARS-CoV and SARS-CoV-2 s2m RNA at increasing concentrations of Mg²⁺ ions. Samples were incubated with indicated concentrations of MgCl₂ following annealing and snap-cooling, then electrophoresed in both TBE (left panel) and TBM (right panel) nondenaturing gels. In the absence of Mg²⁺ ions, both SARS-CoV and SARS-CoV-2 exist in a primarily monomeric state. However, in the presence of Mg²⁺ ions, both RNAs form kissing complex interactions, which are converted to a stable, extended duplex in SARS-CoV-2. In both panels arrow 1 indicates the monomeric s2m, arrow 2 indicates the dimeric kissing dimer conformation and arrow 3 indicates the dimeric duplex conformation.

FIG. 3 is a schematic representation of SARS-CoV-2 s2m kissing complex intermediate formation, which is converted to a thermodynamically stable duplex. The GUAC loop palindrome within the loop region of s2m is highlighted in black for both motifs.

FIG. 4 shows a nondenaturing gel electrophoresis TBE gel demonstrating miR-1307-3p binding to both SARS-CoV and SARS-CoV-2 s2m motifs. Arrows represent the migration of the miR-1307-3p monomer (1), s2m RNA monomer (2), miR-1307-3p dimer (3), RNA:miRNA complex (4), and miRNA:RNA:miRNA complex (5). S.A. indicates control samples that were slow annealed.

FIG. 5 shows a nondenaturing gel electrophoresis TBE gel demonstrating miR-1307-3p binding to the monomeric s2m motifs of SARS-CoV and SARS-CoV-2 in the absence of Mg²⁺ ions. The conversion to a primarily dimeric population of SARS-CoV s2m in the presence of Mg²⁺ ions results in decreased ability for binding to miR-1307-3p. Arrows represent the migration of the miR-1307-3p monomer (1), s2m RNA monomer (2), s2m RNA:miRNA complex (3), and miRNA: s2m RNA:miRNA complex (4).

FIG. 6 shows a nondenaturing gel electrophoresis of SARS-CoV-2 s2m binding to FANA-1307. Monomeric s2m is indicated by arrow 1 of the TBE gel. As increasing concentrations of FANA-1307 (0.25, 0.5, 1, 2, 3, and 4 μM) are added, increased band intensity of two distinguishable bands is seen (arrows 2 and 3). Arrow 2 falls slightly above the 50 molecular weight marker, supporting the 41-nt s2m binds to one FANA-1307 (22-nt). Arrow 3 is seen in the middle of the 50 and 100 marker, suggesting this band corresponds to two FANA-1307s binding to one s2m (a total size of 85 nts) The FANA-1307 exists as a dimer at 44 nts.

FIGS. 7A-7B show schematic representations of proposed miR-760-3p interactions with the 3′ UTR Terminus of the SARS-CoV-2 genome, created using RNAStructure and Structure Editor software packages. FIG. 7A: The miR-760-3p sequence is highlighted in gray, while that of the 3′ UTR Terminus is highlighted in black. FIG. 7B: The miR-760-3p sequence is highlighted in gray, while that of the 3′ UTR Terminus is highlighted in black and the 3′ UTR 100-117 is highlighted in white.

FIG. 8 shows a nondenaturing gel electrophoresis TBE (left panel) and TBM (right panel) gels demonstrating miR-760-3p binding to 3′ UTR Terminus sequence. Control samples for each sequence (lanes 1-3) were annealed and snap-cooled. In lanes 4-8, samples contain 3′ UTR 100-117 and 3′ UTR Terminus, annealed and snap-cooled together, prior to addition of miR-760-3p. Lane 9 (1:1:1*) contains all components, annealed and together in equal ratios. Arrows correspond to the following bands in the TBM gel: (1) monomeric 3′ UTR 100-117, (2) monomeric miR-760-3p, (3) monomeric 3′ UTR Terminus, (4) complex between one 3′ UTR 100-117 and one 3′ UTR Terminus, (5) dimeric miR-760-3p, (6) complex between one 3′ UTR Terminus and one miR-760-3p, (7) complex between two miR-760-3p, each bound to one copy of 3′ UTR Terminus.

FIGS. 9A-9B show nondenaturing gel electrophoresis TBE (left panels) and TBM (right panels) demonstrating DY547-miR-760-3p binding to the 3′-UTR Terminus sequence. FIG. 9A: The DY547-miR-760-3p in the experiments was tracked through a fluorescent signature (taken with a 600 nm imaging channel). FIG. 9B: An overlay of the fluorescent image with a SYBR gold stain image reveals the presence of DY547-miR-760-3p in the previously observed higher molecular weight complexes at ˜80 and ˜160 nt, predicted to be a 3′-UTR Terminus:miR-760-3p complex and a dimer of this complex, respectively.

FIG. 10 shows steady-state fluorescence spectroscopy of miR-760-3p binding to the 3′-UTR 100-177:Terminus duplex as shown by the normalized fluorescent emission at 445 nm following sequential titration of miR-760-3p in 12.5 nM increments to a final titrant concentration of 250 nM.

FIGS. 11A-11B are schematic representations of the proposed miR-760-3p interactions with the 3′-UTR Terminus (FIG. 11A) in comparison with the FANA-760 analog (FIG. 11B), created using RNAstructure and StructureEditor software packages. The 3′-UTR Terminus is highlighted in white, miR-760-3p is highlighted in black, and FANA-760 is highlighted in gray.

FIG. 12 shows a nondenaturing gel electrophoresis demonstrating the binding of FANA-760 to the 3′-UTR Terminus. The FANA-760 is shown to bind to the 3′-UTR Terminus in the presence (TBM, right panel) and absence of Mg²⁺ (TBE, left panel).

FIG. 13 shows steady-state fluorescence spectroscopy of the FANA-760 binding to the 3′-UTR Terminus, as shown by the normalized fluorescent emission at 445 nm following titration of FANA-760 to a final titrant concentration of 250 nM.

FIGS. 14A-14B are schematic representations of the DIS-s2m extended sequence (FIG. 14A) containing the s2m motif (dark gray), and extension of the bottom stem loop (gray), and miR-34a-5p binding site (gray), created using RNAstructure and StructureEditor software packages. Bound miR-34a-5p to the DIS-s2m extended sequence (FIG. 14B) shows the extension of the miR-34a-5p binding interactions through the binding initiation site (gray) into the terminal stem loop of the s2m motif. The miR-34a-5p is highlighted in black.

FIG. 15 shows a nondenaturing gel electrophoresis showing the miR-34a-5p binding experiments with the DiS-s2m extended. The miR-34a-5p is shown to bind in a 1:1 duplex structure with the DiS-s2m extended, and can form in both the presence (TBM, right panel) and absence of Mg²⁺ (TBE, left panel).

### DETAILED DESCRIPTION OF THE PREFERRED EMBODIMENTS

Unless defined otherwise, all technical and scientific terms used herein have the same meaning as is commonly understood by one of skill in the art to which this invention belongs. All publications and patents referred to herein are incorporated by reference.

As used herein, the articles “a” and “an” may refer to one or to more than one (e.g. to at least one) of the grammatical object of the article.

As used herein, “about” may generally refer to an acceptable degree of error for the quantity measured given the nature or precision of the measurements. Example degrees of error are within 5% or 1% of a given value or range of values.

Embodiments described herein as “comprising” one or more features may also be considered as disclosure of the corresponding embodiments “consisting of” and/or “consisting essentially of” such features.

As used herein, a “subject” is an animal which is capable of suffering from or afflicted with a disease. In some embodiments, the subject is a mammal, for example, a human, non-primate, dog, cow, horse, pig, sheep, goat, cat, mouse, rabbit, rat, or transgenic non-human animal.

As used herein, the term “treat” means to relieve, reduce or alleviate at least once symptom of a disease in a subject.

As used herein, a “therapeutically effective amount” of a therapeutic agent, or combinations thereof, is an amount sufficient to treat disease in a subject.

As used herein, the term “PNA” means a peptide nucleic acid. PNAs are nucleic acid analogs in which the sugar phosphate backbone of natural nucleic acid has been replaced by a synthetic peptide backbone. In some embodiments, gamma PNAs are employed. PNAs and gamma PNAs are described in Oyaghire, S. N. et al. (2016) Biochemistry, 55, 1977-1988, which is hereby incorporated by reference in its entirety. In some embodiments, the PNA or gamma PNA further comprises a C-terminal lysine residue.

As used herein, the term “LNA” means a locked nucleic acid. A LNA is a modified RNA nucleotide in which the ribose moiety is modified with an extra bridge connecting the 2′ oxygen and 4′ carbon.

As used herein, the term “cytokine storm” means a severe immune reaction in which the body releases too many cytokines into the blood too quickly. A cytokine storm can occur as a result of an infection, autoimmune condition, or other disease.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Provided is a novel therapeutic target for SARS-CoV-2 in the form of an s2m motif. The s2m motif is involved in genomic RNA dimerization, and it binds two or more molecules of a cellular microRNA, miR-1307-3p, a regulatory microRNA that has been proposed to be involved in the innate immune response, which is upregulated in a variety of cancers. Moreover, the s2m dimerization impacts its ability to interact with miR-1307-3p.

Also provided is a novel therapeutic target for SARS-CoV-2 in the form of the 3′-UTR Terminus which binds to a host cellular microRNA, miR-760-3p, which is proposed to regulate the translation of interleukin 6 (IL6), one of the cytokines found at the eye of the cytokine storm.

Additionally, provided is a novel therapeutic target for SARS-CoV-2 in the form of DIS-s2m extended, which binds the host cellular microRNA, miR-34a-5p, which is proposed to regulate the expression of IL6 through regulatory pathways and ultimately influence the cytokine storm.

MicroRNAs (miRNAs) are short, noncoding RNAs that regulate the translation of more than sixty percent of all mammalian genes by guiding the RNA-induced silencing complex to various target messenger RNAs (mRNAs). Viruses have been shown to “hijack” various miRNAs for their own benefit to either aid in their replication and progression through the viral life cycle, or to alter the miRNA interactions with their cognate host mRNAs and thus, affect the host cellular response to the viral infection. A bioinformatics analysis of the both miR-1307-3p, miR-760-3p, and miR-34a-5p reveals a variety of potential cellular mRNA targets encoding for proteins involved in the innate immune response, some of which have been specifically shown to be involved in the cytokine storm immune response.

It has been contemplated that SARS-CoV-2 has led to a cytokine storm in a number of COVID-19 patients, which in turn leads to respiratory failure from acute respiratory distress syndrome (ARDS), the leading cause of mortality in COVID-19. SARS-CoV-2 contains two binding sites for miR-1307-3p within its s2m motif. This motif mutated from the s2m motif of SARS coronavirus (SARS-CoV), the virus which caused the 2002-2003 SARS outbreak, creating a two-nucleotide variation. A bioinformatics analysis of the s2m motif within SARS-CoV-2 determined that from December 2019 until Jul. 1, 2020, the motif that previously was invariant in some of its nucleotides among different coronaviruses, has evolved into at least 15 mutants. Moreover, the SARS-CoV-2 Delta variant which is currently responsible for 99% of the COVID-19 infections in U.S., has been found to have a G to U mutation at position 15 within the s2m motif. Experimental tests of the s2m G15U mutant have demonstrated that it is dimerizing less than the wild type SARS-CoV-2 (the December 2019, Wuhan strain) and has lower miRNA-1307-3p binding affinity.

SARS-CoV-2 uses its s2m motif to bind the cellular miR-1307-3p, wherein the process is fine-tuned by the ability of the s2m motif to dimerize. The “hijacking” of miR-1307-3p by SARS-CoV-2 prevents this miRNA from performing its normal cellular function which is potentially to repress the translation of various interleukins (IL18, CCLS) and interleukin receptors (IL6R, IL10RA, IL10RB, IL2RB, IL17RA, IL12RB2, and the like), and interferon alpha receptor (IFNAR) whose upregulated levels have been linked to the onset of a cytokine storm. Without wishing to be bound by theory, another potential role of miRNA-1307-3p in the lifecycle of SARS-CoV-2 relates to mechanisms of viral entry and propagation. For example, recognition of the ACE2 receptors on ATII lung epithelial cells by the SARS-CoV-2 S protein is generally regarded as the first step in viral entry and subsequent infection. However, bioinformatic analyses have revealed that multiple other receptor proteins, including transmembrane protease serine 2 (TMPRSS2) and glucose regulating protein 78 (GRP78), contain binding sites for the S protein of SARS-CoV-2 as well. The latter of these proteins, GRP78, has been suggested to play a role in attachment of the SARS-CoV-2 S protein. Interestingly, miRNA-1307-3p has been proposed to indirectly reduce the GRP78 production, among other functions, by regulating the translation of proteins involved in glycogenesis. Given this correlation, it is possible that the sequestration of miRNA-1307-3p in lung epithelial tissue by the SARS-CoV-2 s2m motif could prevent it from inhibiting the GRP78 production, which would benefit viral entry. Thus, targeting the s2m motif directly and/or its dimerization by either small molecules or antisense molecules (FANAs, PNAs, LNAs, and the like) will prevent and/or disrupt its interactions with miR-1307-3p, which in turn will release this miRNA to perform its normal cellular function, potentially preventing the onset of a cytokine storm and preventing viral entry.

The SARS-CoV-2 virus uses its 3′-UTR Terminus (29,828-29,870) to bind another cellular microRNA, miR-760-3p, “hijacking” this microRNA from performing its normal cellular functions. Notably miR-760-3p has been experimentally demonstrated to regulate the translation of IL6, a cytokine that has been directly linked to the cytokine storm in severe COVID-19 cases. Interestingly, miR-760-3p downregulates translation of IL6, whereas miR-1307-3p is proposed to downregulate the translation of the IL6 receptors. It is demonstrated herein that the SARS-CoV-2 virus binds both miRNAs, impairing their function in the regulation of the IL6-IL6R immune response.

The SARS-CoV-2 virus uses a predicted binding site contained in an extended 3′-UTR s2m motif (genome positions 29,768-29,790) to bind cellular microRNA, miR-34a-5p, “hijacking” this microRNA from performing its normal cellular functions. Interestingly, miR-34a-5p has been shown to regulate IL6 expression through the JAK/STAT pathway. Direct translational regulation of plasminogen activator inhibitor-1 (PAI-1) by miR-34-5p triggers a downregulation of toll-like receptor 4 (TLR4) and subsequent downregulation of IL6. The combined roles of miR-1307-3p, miR-760-3p, and miR-34a-5p suggest regulation of IL6 and IL6R through translational inhibition and inhibition through cellular signaling pathways. It is demonstrated herein that the SARS-CoV-2 virus binds all three miRNAs, allowing the virus to “hijack” host cellular regulation and ultimately impairing cellular control of the IL6-IL6R immune response.

Provided is an s2m motif as a novel therapeutic target for SARS-CoV-2. This motif and its ability to dimerize and/or interact with miRNA-1307-3p is targeted. The dimerization of the motif and, in turn its binding with miRNA-1307-3p, is controlled or fine-tuned to prevent and/or disrupt binding of microRNA with the s2m motif, thereby allowing the miR-1307-3p to perform its normal cellular function and preclude the onset of a cytokine storm. The method of targeting the s2m motif and fine-tuning dimerization includes introducing small molecules or antisense molecules (FANAs, PNAs, LNAs, etc) to the s2m motif.

Additionally provided is the 3′-UTR Terminus as a novel therapeutic target for SARS-CoV-2. This sequence and its ability to interact with miR-760-3p is targeted, releasing miR-760-3p to regulate the translation of IL6 and potentially prevent the onset of a cytokine storm in severe COVID-19 cases.

Also provided is DIS-s2m extended as a novel therapeutic target for SARS-CoV-2. This sequence and its ability to interact with miR-34a-5p is targeted, releasing miR-34a-5p to regulate the translation of IL6 and potentially prevent the onset of the cytokine storm in severe COVID-19 cases.

### EXAMPLES

The following Examples are not intended to be limiting. In some of the following experiments, nondenaturing gel electrophoresis was used to demonstrate SARS-CoV-2 genome dimerization is mediated through the 41-nucleotide s2m motif and the s2m region was found to bind cellular miR-1307-3p, an interaction which was modulated by the s2m dimerization. Additionally, the same techniques were used to demonstrate that the SARS-CoV-2 3′-UTR Terminus binds the cellular miR-760-3p, and that an the DIS-s2m extended motif binds the cellular miR-34a-5p. The results implicate the SARS-CoV-2 s2m motif, its extended version DIS-s2m and the 3′-UTR Terminus as potential therapeutic targets for COVID-19, as well as future coronavirus-related disease outbreaks.

### Materials and Methods

### RNA Synthesis

RNA oligonucleotides of SARS-CoV and SARS-CoV-2 s2m motifs (41-nucleotides; Table 1), 3′ UTR 100-117 (18-nucleotides; Table 1), 3′ UTR Terminus (42-nucleotides; Table 1), miR-1307-3p (22-nt), miR-760-3p (20-nt; Table 1) and miR-34a-5p (20 nt) were chemically synthesized by Dharmacon, Inc (Lafayette, Colo., USA). Lyophilized RNA was re-suspended in 10 mM cacodylic acid, pH 6.5, and diluted in ½× Tris-Boric Acid (TB) buffer prior to annealing and snap-cooling with dry ice.

TABLE 1
RNA oligonucleotide sequences used
in this study. Nucleotide variances
between SARS-CoV and SARS-CoV-2
are highlighted in italic and
underlined.

| SEQ | | |
|---|---|---|
| ID | | |
| NO: | Name | Sequence |
| 10 | SARS- | 5′ UUCAUCGAGGCCA |
| | CoV s2m | CGCGGAGUACGAU |
| | | CGAGGGUACAGUGAA 3′ |
| 11 | SARS- | 5′ UUCACCGAGGCCA |
| | CoV-2 | CGCGGAGUACGAUCG |
| | s2m | AGUGUACAGUGAA 3′ |
| 12 | miR- | 5′ ACUCGGCGUGGCG |
| | 1307-3p | UCGGUCGUG 3′ |
| 13 | SARS- | 5′ CUUUAAUCUCAC |
| | CoV-2 | AUAGCA 3′ |
| | 3′ UTR | |
| | 100-117 | |
| 14 | SARS- | 5′ UGCUAUCCCCAU |
| | CoV-2 | GUGAUUUUAAU |
| | 3′ UTR | AGCUUCUUAGGAGAAUGAC 3′ |
| | Terminus | |
| 15 | miR- | 5′ CGGCUCUGGGUC |
| | 760-3p | UGUGGGGA 3′ |
| 16 | SARS- | 5′ CAUUUUCACCGA |
| | CoV-2 | GGCCACGCGGAGUACGAUCGA |
| | DIS-s2m | GUGUACAGUGAACAAUGCUAG |
| | Extended | GGAGAGCUGCCUA3′ |
| 17 | miR-34a- | 5′ UGGCAGUGUCU |
| | 5p | UAGCUGGUUGU 3′ |
| 4 | FANA- | 5′ ACUCCGCGUGG |
| | 1307 | CCUCGGUCGUG 3′ |
| 5 | FANA- | 5′ AAGAAGCUAUU |
| | 760 | AAAAUCACAUGGGGA 3′ |

### Nondenaturing Gel Electrophoresis

To study RNA dimerization, samples were diluted from stock solutions to a concentration of 1 μM, followed by annealing at 95° C. and immediate snap-cooling. Samples were then incubated in the presence of 1-10 mM MgCl₂ for 60-minutes to induce kissing complex formation. RNA was electrophoresed on 12% nondenaturing polyacrylamide gels at 75 V and 4° C. in the presence of either Tris-Boric Acid-EDTA (TBE; 2-hours) or Tris-Boric-Acid-MgCl₂ (TBM; 4-hours) buffers. TBM gels were prepared with 5 mM MgCl₂ both in the gel and buffer solutions.

The miRNA and FANA binding experiments were performed similarly, at constant RNA [1 μM] and MgCl₂ [1 mM] concentrations. Samples were annealed, snap-cooled, and incubated in the presence of both MgCl₂ and indicated miRNA and FANA concentrations for 60-minutes. Both TBE and TBM gels were run as described above.

### Steady-State Fluorescence Spectroscopy

Quantitative analysis of the miRNA and FANA binding affinities (K_{d}) was determined through the quenching of a pyrollo-cytosine fluorescent tag. Samples were diluted from stock concentrations to a concentration of 150 nM, followed by annealing at 95° C. and immediate snap-cooling. An initial intensity of the fluorescent emission was taken at 445 nm, followed by titration of respective miRNA or FANA in 12.5 nM or 25 nM increments. Immediately following each addition, the fluorescent emission was recorded and plotted. Titrations were completed to a final titrant concentration of 250 nM. The collected emissions were normalized and fit to determine a K_{d} value for the predicted binding interactions.

### Example 1: SARS-CoV-2 Dimerization is Mediated Through the s2m Motif

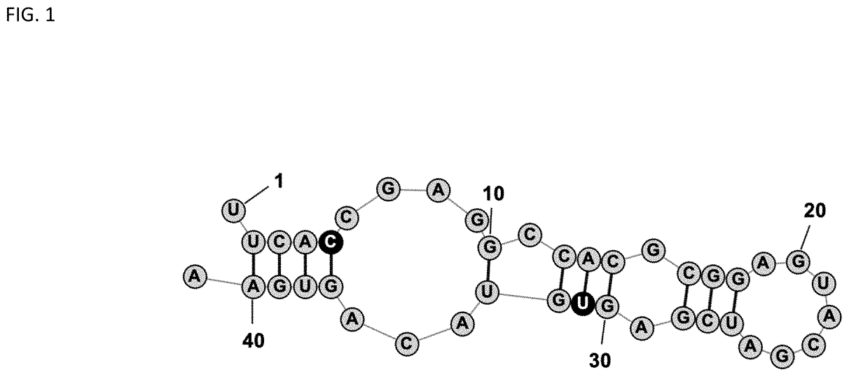

Highly conserved regions within RNA(+) genomes have previously been shown to mediate genome dimerization via the formation of kissing complex intermediates that are converted to stable, extended duplexes by viral proteins. In the case of both hepatitis C virus (HCV) and HIV type 1 (HIV-1), this is mediated through a palindromic 4- to 6-nucleotide loop sequence towards the 3′ and 5′ end of the genome, respectively. Here, there was investigated a similar function mediated by a palindromic 4-nucleotide (GUAC) loop sequence located within the highly conserved s2m motif in the 3′ UTR hypervariable region of SARS-CoV-2 (FIG. 1).

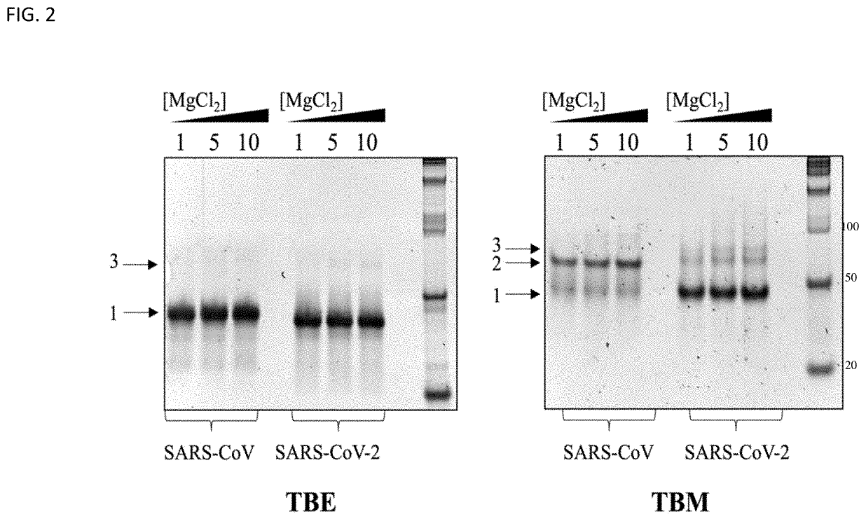

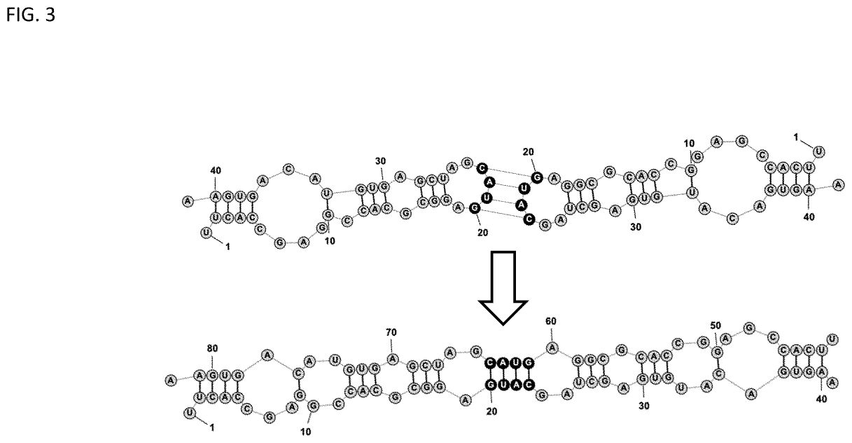

The SARS-CoV-2 s2m motif varies from that of SARS-CoV (Table 1) by only two nucleotides: a U to C mutation at position 5 and a G to U mutation at position 31 (shown in black in FIG. 1). Given this variation, the inventors also sought to investigate how these variances affect dimerization in SARS-CoV-2 compared to SARS-CoV. Using nondenaturing gel electrophoresis, kissing complex formation was monitored at increasing concentrations of Mg²⁺ ions, which are known to be essential in the formation of these RNA-RNA interactions (FIG. 2). Upon chelation of Mg²⁺ ions in the TBE gel (FIG. 2, left panel), both SARS-CoV and SARS-CoV-2 exist primarily in a monomeric state, with a faint dimeric band present at higher concentrations of Mg²⁺ for SARS-CoV-2. The dimeric band likely corresponds to a stable, extended duplex (FIG. 3, bottom) as a kissing complex structure (FIG. 3, top) would dissociate due to the lack of Mg²⁺ ions in the TBE gel. Conversely, in the presence Mg²⁺ ions, dimeric conformations were evident in both SARS-CoV and SARS-CoV-2 (FIG. 2, right panel). At all concentrations of Mg²⁺ ions, SARS-CoV existed primarily in a dimeric state (FIG. 2, right panel, lanes 1-3, arrow 2), whereas SARS-CoV-2 shows two bands corresponding to dimer complexes (FIG. 2, right panel, lanes 3-6, arrows 2 and 3), but also a strong intensity monomer band (FIG. 2, right panel, lanes 3-6, arrow 1). As the concentration of Mg²⁺ ions was increased in the SARS-CoV-2 s2m RNA, the monomer band decreased in intensity and a concomitant increase in the top dimer band intensity occurred. When the Mg²⁺ ions were chelated by EDTA in the TBE gel, SARS-CoV appears monomeric (FIG. 2, left panel, lanes 1-3, arrow 1), whereas a faint single dimer band is present in SARS-CoV-2 (FIG. 2, left panel, lanes 4-6, arrow 3). Because this dimer band is present even in the absence of Mg²⁺, it was attributed to the duplex conformation. Thus, it was concluded that the dimer bands present in the TBM gel correspond to the kissing complex in SARS-CoV and to a kissing complex intermediate (arrow 2) which is converted to a stable duplex (arrow 3) (FIG. 3).

While the role of the s2m motif within coronaviruses remains elusive, it has been proposed to be involved in viral replication, hijacking of host protein synthesis, and play roles in RNA interference pathways. Given the conservation of the s2m motif among various viral families, it was proposed that the motif also functions in genome dimerization and subsequent homologous recombination between viral genomes, resulting in novel viruses. Homologous recombination of bat and pangolin coronaviruses is believed to have been the origination of SARS-CoV-2 and triggered its ability to transmit to humans.

### Example 2: S2m Motif Interacts with Multiple Copies of Cellular miR-1307-3p

Bioinformatic analysis of microRNAs targeting the SARS-CoV-2 s2m RNA revealed that cellular miR-1307-3p, upregulated in a variety of cancers, potentially has multiple binding sites within the s2m motif. This particular microRNA additionally targets the mRNAs of various interleukins and interleukin receptors which have been linked to the innate cytokine storm, a common hallmark of COVID-19, under normal conditions. Given the potentially significant role of this microRNA in COVID-19 pathogenesis, its binding to the SARS-CoV-2 s2m region was investigated.

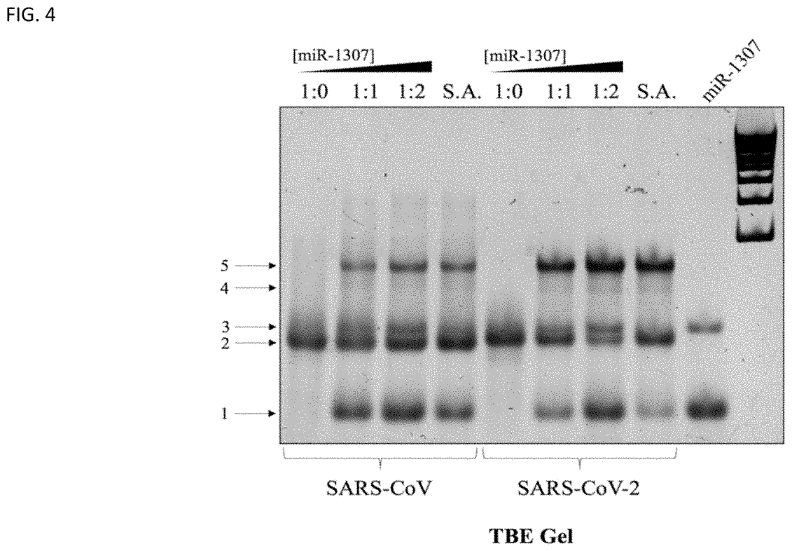

SARS-CoV and SARS-CoV-2 s2m RNA were incubated in the presence of increasing concentrations of miR-1307-3p (1:0, 1:1, 1:2) and electrophoresed on a 12% nondenaturing TBE gel (FIG. 4). Both s2m RNAs were found to interact with miR-1307-3p, with the SARS-CoV-2 RNA:miRNA complex band having a greater intensity compared to the SARS-CoV RNA:miRNA complex bands (FIG. 4, arrow 5), as well as a concomitant decrease in the SARS-CoV-2 s2m monomer band (FIG. 4, arrow 2). This finding supported the hypothesis that the s2m motif of coronaviruses may act to sequester host microRNAs, as has been studied with various other viruses, in order to inhibit their normal cellular functions. In the case of miR-1307-3p, this sequestration could potentially result in an upregulation of its target mRNA products, including various interleukins and interleukin receptors thought to be involved in the onset of a cytokine storm found in severe COVID-19 patients. By targeting and inhibiting this interaction via therapeutic drugs, antisense RNAs or PNAs, miR-1307-3p could potentially return to performing its normal cellular functions and prevent the onset of fatal COVID-19 symptoms, such as acute respiratory distress syndrome (ARDS) caused by the cytokine storm.

### Example 3: S2m Dimerization Impacts miR-1307-3p Binding Ability

It was observed that SARS-CoV binds less miR-1307-3p than SARS-CoV-2 does (compare band intensities shown by arrow 5 in FIG. 4, lanes 2, 3 and 6, 7). To determine if this difference in binding is due to an intrinsic different binding affinity of the monomeric s2m motif between the two viruses or due to the different dimerization properties of the two viruses there were performed miR-1307-3p binding experiments in the absence of MgCl₂, where both the SARS-CoV and SARS-CoV-2 s2m motifs are monomeric.

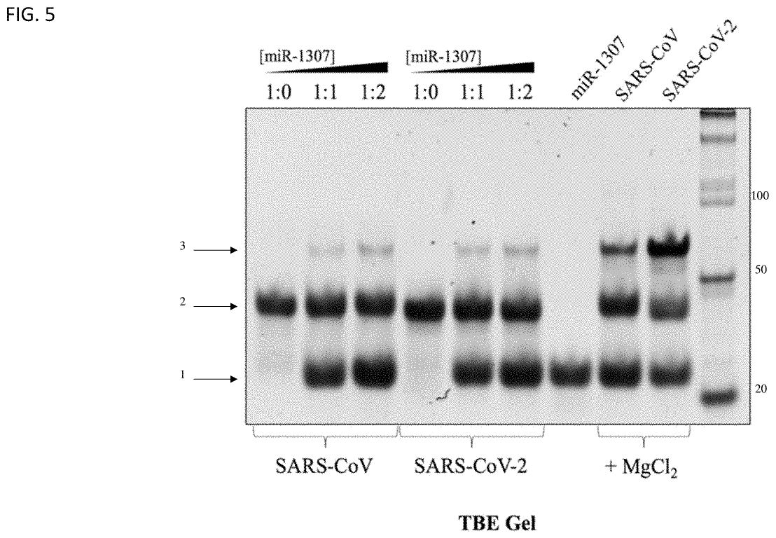

Both SARS-CoV and SARS-CoV-2 s2m RNAs were incubated in the absence of Mg²⁺ ions with the microRNA and electrophoresed on a 12% TBE nondenaturing gel (lanes 1-6, FIG. 5). While both s2m motifs bound miR-1307-3p, in the absence of Mg²⁺ there were no distinguishable differences between SARS-CoV and SARS-CoV-2 RNA:miRNA complex bands, indicating similar binding affinities to the monomeric s2m. In contrast, incubation of each s2m motif with 1 mM MgCl₂, which, as showed previously induces kissing complex formation, followed by incubation with miR-1307-3p, resulted in increased intensity of the SARS-CoV-2 RNA:miR-1307-3p complex band compared to that of SARS-CoV-miR-1307-3p (FIG. 5, lanes 8 & 9). Given that the SARS-CoV-2 s2m motif exists primarily in its monomeric conformation in the presence of MgCl₂ (FIG. 3), it is proposed that the greater intensity of the SARS-CoV-2 RNA:miR-1307-3p complex band (FIG. 5, lane 9) is due to the increased population of monomeric s2m available for miRNA binding. In contrast, SARS-CoV s2m exists primarily in its dimeric kissing complex conformation in the presence of MgCl₂ (FIG. 2), which prevents similar interactions with miR-1307-3p during sample incubation (FIG. 5, lane 8). Incubation of both SARS-CoV and SARS-CoV-2 s2m motifs with Mg²⁺ ions aids in the formation of RNA:miRNA duplexes (FIG. 5, lanes 8 & 9), as indicated by increased complex band intensities compared to those in the absence of Mg²⁺ ions (FIG. 5, lanes 1-6).

The results from these studies indicate that s2m dimerization affects its interactions with miR-1307-3p, due to the shift in equilibrium between monomeric RNA and homodimeric dimers formed by the motif. SARS-CoV s2m forms a kinetically-stable kissing complex in the presence of Mg²⁺ ions, whereas SARS-CoV-2, under similar conditions, exists primarily in its monomeric state (FIG. 2). Increased RNA:miRNA complex band intensities are seen with SARS-CoV-2 compared to SARS-CoV, due to the increased population of monomeric s2m available for microRNA binding. Without wishing to be bound by theory, miRNA binding to the s2m motif is controlled by a fine-tuned mechanism, impacted by motif dimerization, which could play a role in the onset of the cytokine storm in severe cases of COVID-19.

### Example 4: FANA Analog of miR-1307-3p to the SARS-CoV-2 s2m

A FANA analog of miR-1307-3p (AUM Biotech) was proposed as a binding inhibitor for cellular miR-1307-3p on the s2m motif:

FANA-1307
[5′ ACUCCGCGUGGCCUCGGUCGUG 3′ (SEQ ID NO: 4)].

This analog was constructed as a perfect complement to the binding sites on the s2m of SARS-CoV-2 to allow for stronger binding interactions.

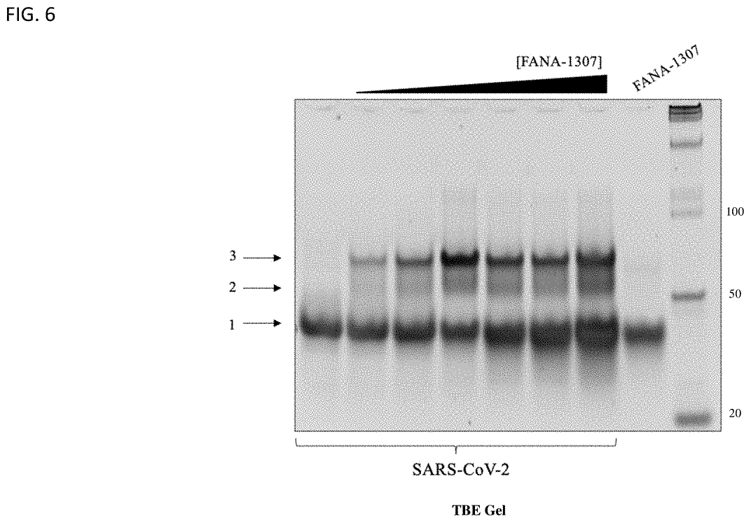

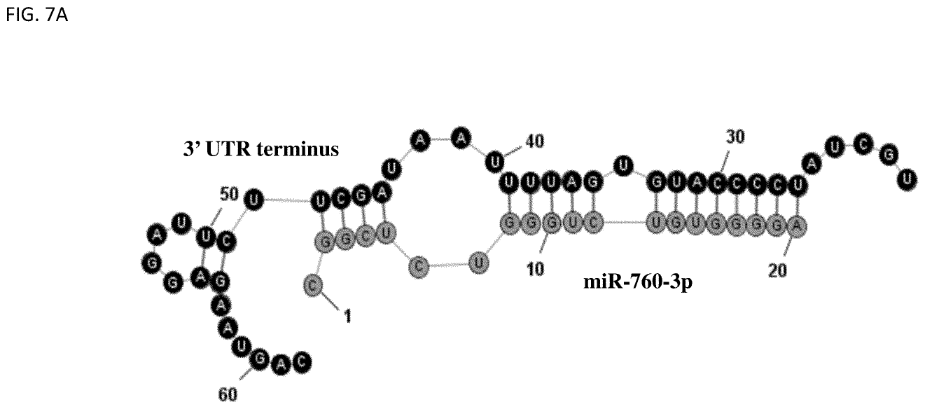

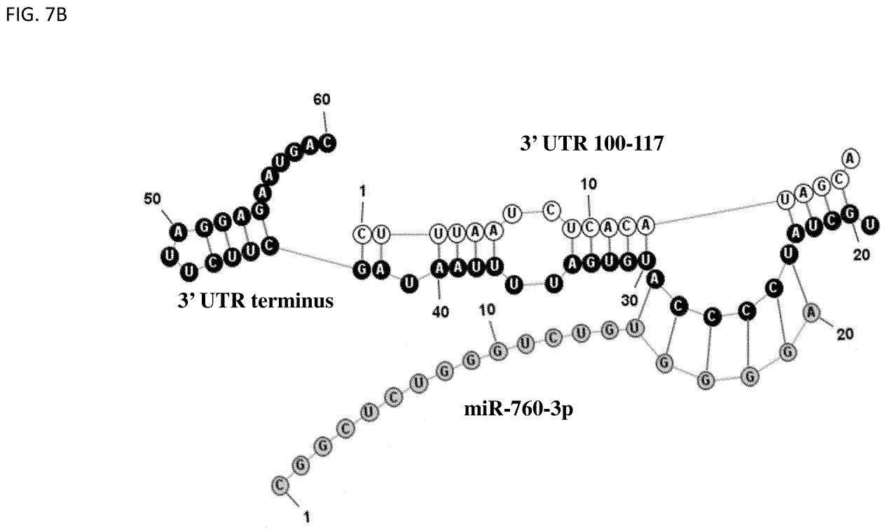

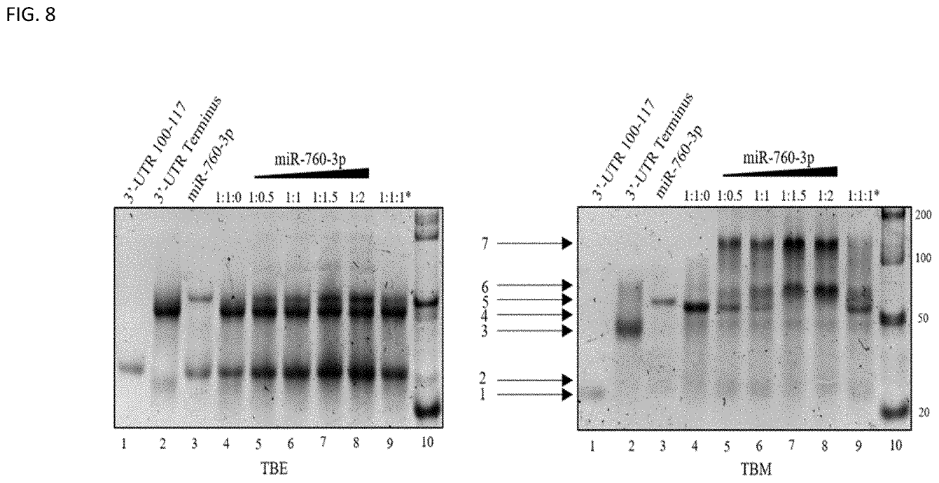

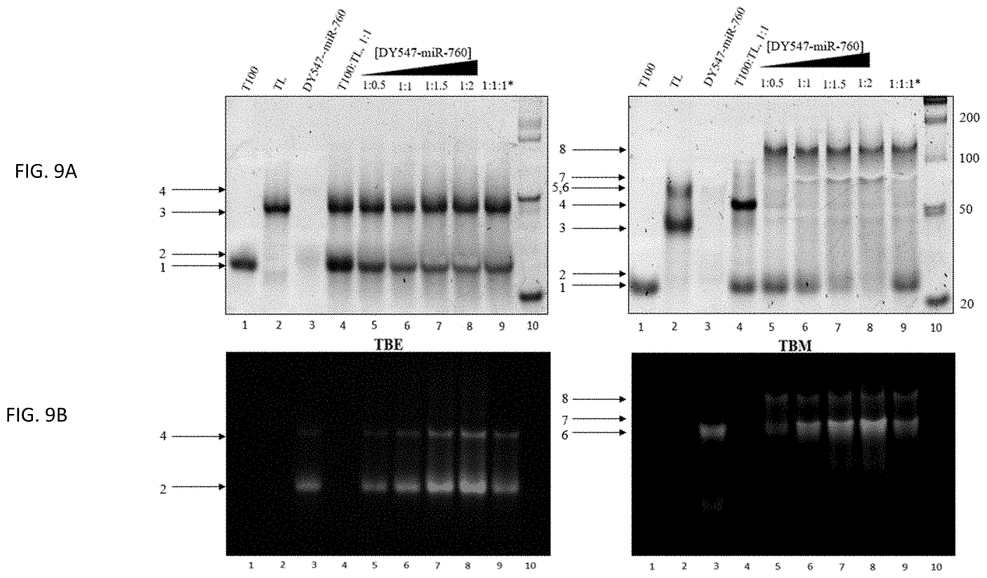

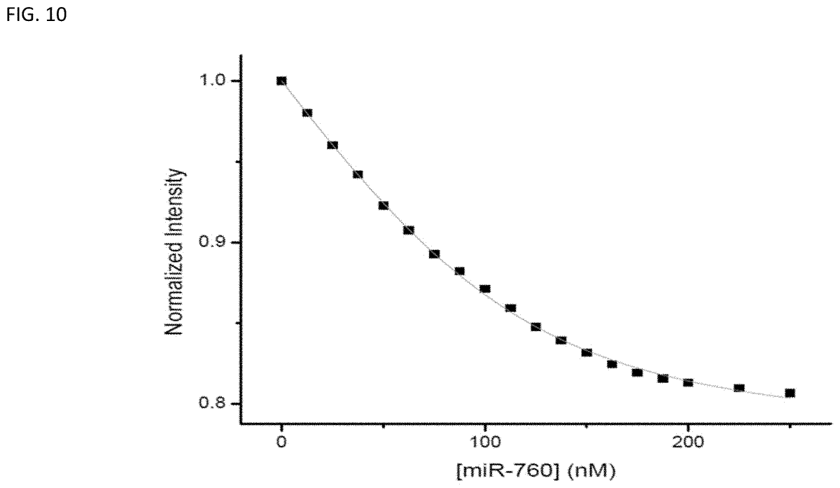

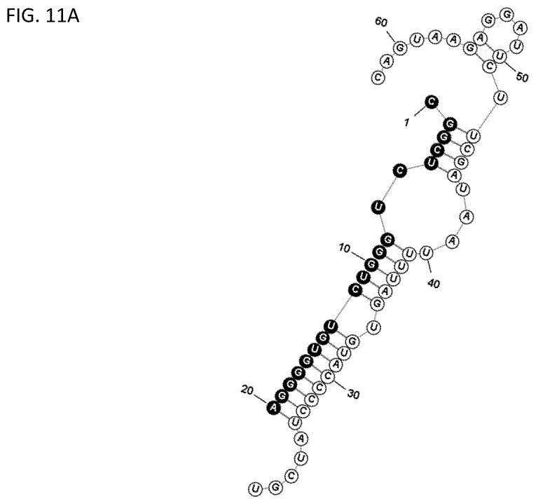

To test this interaction, FANA-1307 experiments were conducted by boiling and snap cooling the s2m RNA then incubated with 1 mM Mg²⁺ for 30 minutes to form the hairpin and kissing dimer. Afterwards, FANA-1307 analog was added in increasing concentration ratios (0.25, 0.5, 1, 2, 3, and 4 μM) and left to incubate at 20° C. for an additional 30 minutes. The samples were electrophoresed at 75V and 4° C. on a native TBE gel for 2 hours. Binding of the FANA-1307 to the s2m is seen through the appearance of two new bands which increase in intensity. This result supports that the s2m can bind one or two FANAs with high affinity (FIG. 6, arrows 2 and 3). Further experimentation will test the ability of the FANA-1307 to disrupt and replace miR-1307-3p binding interactions on the s2m, providing a potential therapeutic to the onset of the cytokine storm.

### Example 5: FANA Analog of miR-760-3p Binds to the 3′ UTR Terminus

It has been determined by the inventors that a FANA analog of the miR-760-3p can serve as a binding inhibitor for the proposed interactions of miR-760-3p with the 3′-UTR Terminus. The FANA analog (AUM Biotech) was designed as a perfect complement to the predicted miR-760-3p binding site on the SARS-CoV-2 3′-UTR:

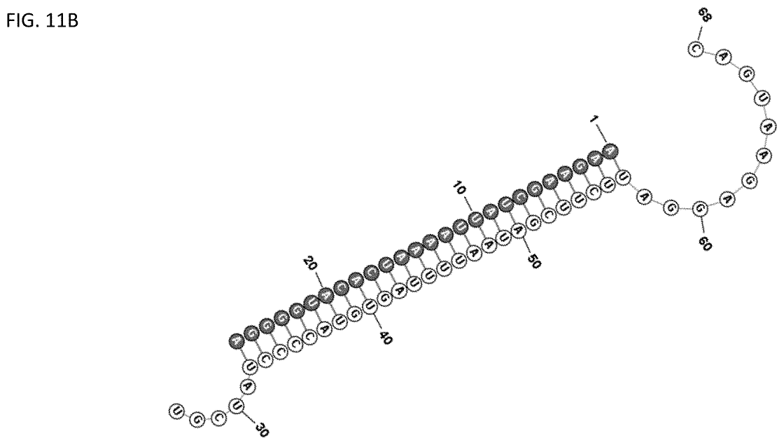

FANA-760
[5′AAGAAGCUAUUAAAAUCACAUGGGGA 3′ (SEQ ID NO: 5)]
(FIG. 11B).

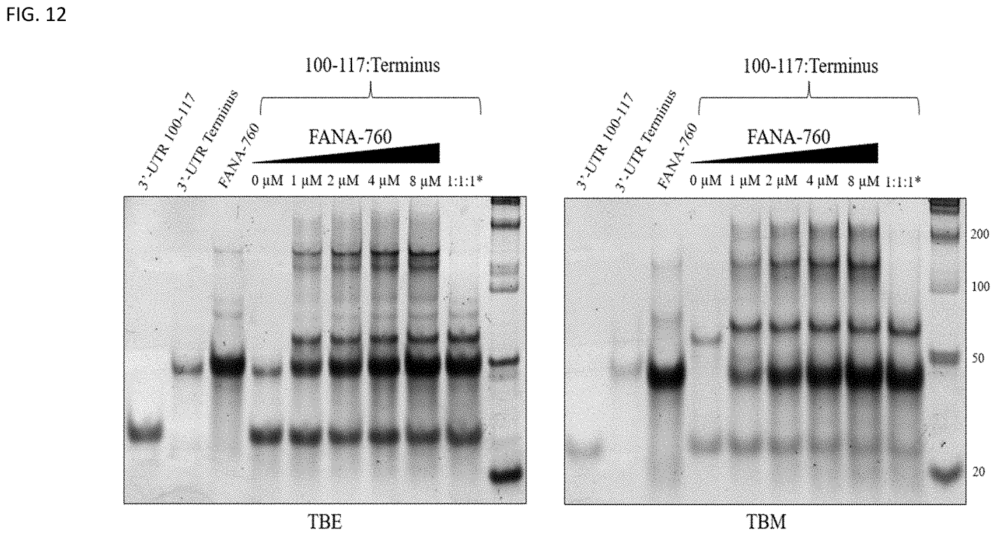

FANA-760 was designed to have perfect sequence complementarity to the proposed miR-760-3p binding site, allowing FANA-760 to serve as a binding inhibitor with a higher binding affinity and overall complex stability. For comparison and homology, FANA-760 binding experiments were performed similarly to the miR-760-3p binding experiments; FANA-760 was incrementally added to a preformed 3′-UTR 100-117:Terminus duplex followed by electrophoresis on TBM and TBE gels. (FIG. 12). FANA-760 shows binding to the 3′ UTR in the presence of Mg²⁺, showing the ability to bind to the preformed 3′-UTR 100-117:Terminus duplex. Significant binding is also seen in TBE, indicating that the binding interactions are stable in the absence of Mg²⁺. Furthermore, the preformed 3′-UTR 100-117:Terminus duplex is shown to dissociate upon chelation of Mg²⁺ in TBE, suggesting that the binding observed is to the 3′-UTR Terminus. Homologous bands between TBE and TBM gels supports that FANA-760 binds to both the preformed 3′-UTR 100-117:Terminus duplex and the 3′-UTR Terminus monomer.

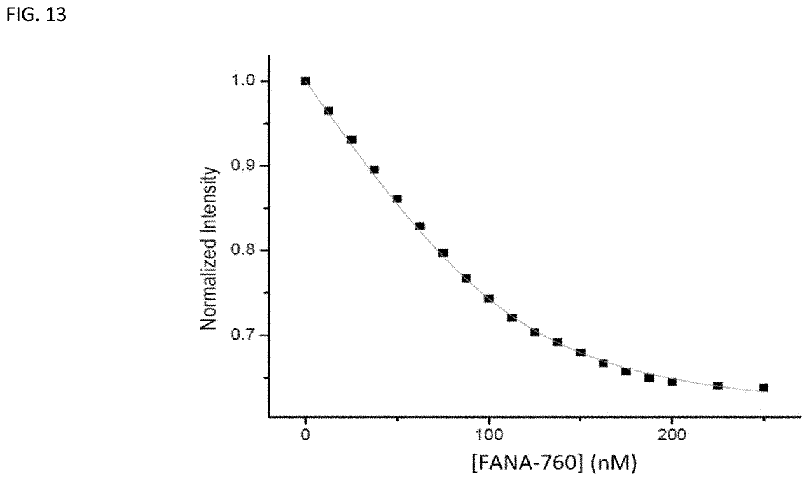

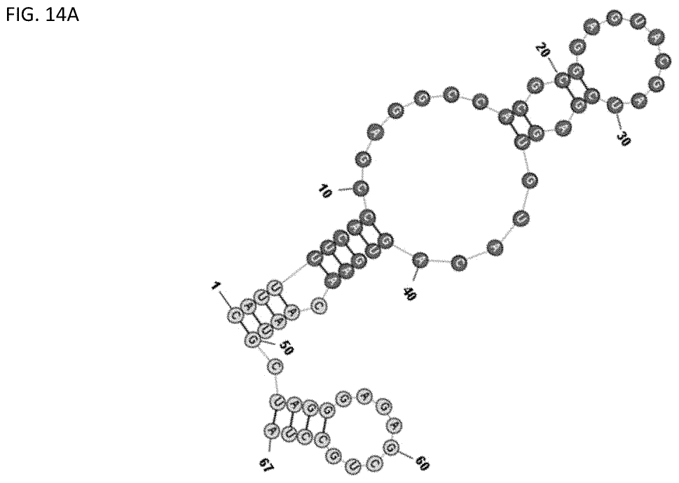

Steady-state fluorescence spectroscopy of FANA-760 binding to the 3′-UTR 100-117:Terminus duplex reveals that FANA-760 binding interactions have an average Kₐ of 16.4±1.9 nM. FANA-760 fluorescence experiments were performed in triplicate. When compared to the miR-760-3p fluorescence experiments, FANA-760 has a lower K_{d} value (miR-760-3p K_{d}: 24.0±4.1 nM), thereby suggesting that FANA-760 has a higher binding affinity than miR-760-3p and supporting the use of FANA-760 as a binding inhibitor (FIG. 13).

### Example 6: Cellular miR-34a-5p Binds to an Extended s2m Motif

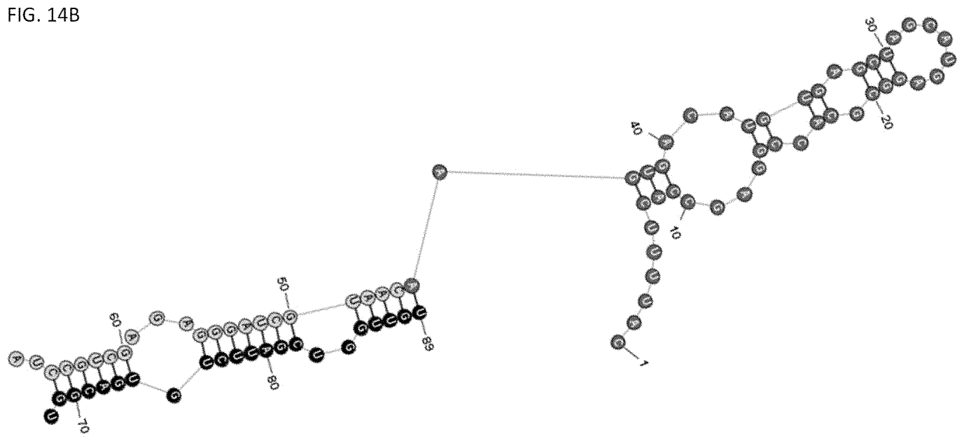

The inventors have determined that the cellular miR-34a-5p has a binding site located downstream of and contained within the s2m motif (FIG. 14). In the native fold, this binding site is seen as a hairpin structure located downstream of the s2m motif, however in close proximity. Considering the predicted binding site for miR-34a-5p extends through the bottom stem of the s2m motif, the inventors have postulated that s2m conformations can influence miR-34a-5p binding. To account for this, miR-34a-5p binding was tested to the DIS-s2m extended sequence, containing the miR-34a-5p binding site and the entire s2m motif.

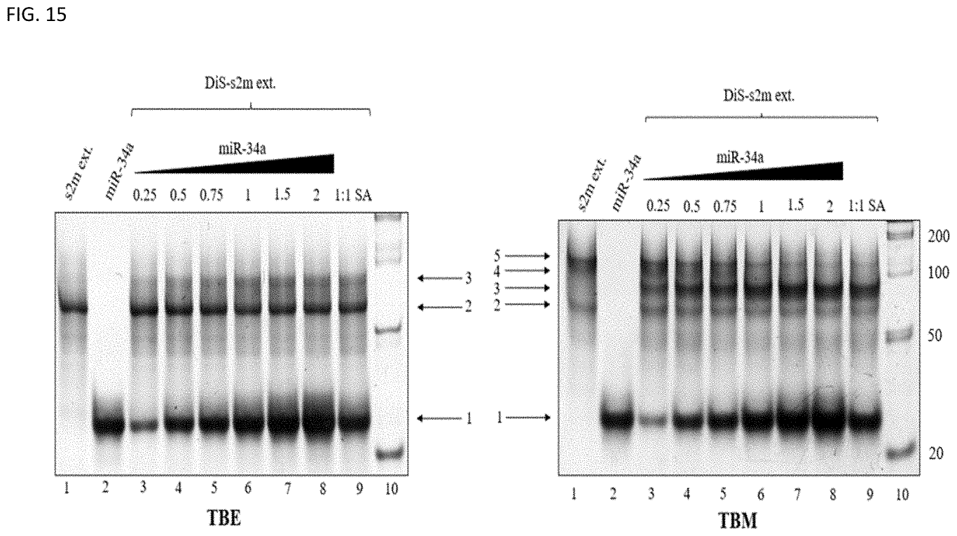

The binding experiments for miR-34a-5p to the DIS-s2m extended were performed similarly to the miR-760-3p binding experiments. Samples were snap cooled and incubated in the presence of Mg²⁺ ions, followed by increasing addition of the miR-34a-5p. The samples were then run on TBE and TBM gels (FIG. 15). In the presence of Mg²⁺, the DIS-s2m extended is shown to exist as a monomer (arrow 2) as well as two dimeric structures (arrows 4 and 5). Upon addition of miR-34a-5p, a 1:1 duplex band of bound miR-34a-5p and DIS-s2m extended is formed (arrow 3) with the concomitant decrease in intensity of dimeric structures. Interestingly, the miR-34a-5p monomer band only shows a slight decrease in intensity, suggesting that the 1:1 miR-34a-5p bound duplexes interact with the dimeric structures, releasing free DIS-s2m extended monomer as duplex structures are dissociated. The 1:1 bound duplex is also shown to form upon chelation of Mg²⁺, suggesting that the complex is stable in the absence of such stabilizing cations.

The role of the s2m motif within coronaviruses remains unresolved, though its conservation among multiple viral families suggests it confers some selective advantage. Without wishing to be bound by theory, there appears to be a role for the s2m motif in RNA dimerization, which could prove vital for key biological mechanisms within the viral life cycle. The results indicate that both SARS-CoV and SARS-CoV-2 s2m motifs form RNA-RNA kissing complexes in the presence of Mg²⁺ ions, which in the case of the novel coronavirus causing the current COVID-19 pandemic, spontaneously converts to a stable, extended duplex. Additionally, the data provides evidence that the s2m motif of both SARS-CoV and SARS-CoV-2 bind to cellular miR-1307-3p, an interaction which is altered by dimerization of the conserved motif. Furthermore, the inventors have shown that the 3′-UTR Terminus of the SARS-CoV-2 genome binds host microRNA, miR-760-3p. The inventors have also identified and demonstrated that an extended s2m motif can bind the host microRNA, miR-34a-5p. The miR-1307-3p is proposed to target the IL6 receptor, and GRP78, whereas the miR-760-3p and miR-34a-5p have been shown to regulate IL6 expression, both of which have been proposed to be involved in the cytokine storm observed in severe COVID-19 cases. The findings that SARS-CoV-2 can hijack these host microRNAs could potentially result in an upregulation of their normal cellular targets, including IL6 and its receptor, as well as various other interleukins and interleukin receptors linked to the cytokine storm seen in patients with severe cases of COVID-19. Moreover, the inventors have demonstrated that antisense nucleic acids to the miRNA binding sites can potentially serve as binding inhibitors and therapeutics to release “hijacked” miRNAs and alleviate the cytokine storm. The results provide a novel mechanism within the SARS-CoV-2 viral life cycle which could be therapeutically targeted by small molecules in the treatment and prevention of COVID-19.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. An agent that binds to a SARS-CoV-2 s2m motif, a SARS-CoV-2 3′-UTR Terminus, or a SARS-CoV-2 DIS-s2m extended sequence.

2. The agent of claim 1, wherein binding of the agent to the SARS-CoV-2 s2m prevents and/or disrupts interaction of the s2m motif with miR-1307-3p.

3. The agent of claim 1, wherein binding of the agent to the SARS-CoV-2 3′-UTR Terminus prevents and/or disrupts interaction of the 3′-UTR Terminus with miR-760-3p.

4. The agent of claim 1, wherein binding of the agent to the SARS-CoV-2 DIS-s2m extended sequence prevents and/or disrupts interaction of the DIS-s2m extended sequence with miR-34a-5p.

5. The agent of claim 1, wherein the agent comprises an engineered peptide nucleic acid (PNA).

6. The agent of claim 5, wherein the PNA is a gamma PNA comprising a sequence selected from the group consisting of:
(SEQ ID NO: 1)
ACUCCGCGUGGCCUCGGUCGUG;
(SEQ ID NO: 2)
AAGAAGCUAUUAAAAUCACAUGGGGA;
and
(SEQ ID NO: 3)
UAGGCAGCUCUCCCUAGCAUUGU.

7. The agent of claim 5, wherein the PNA is a gamma PNA comprising a C-terminal lysine residue and comprising a sequence selected from the group consisting of:
(SEQ ID NO: 1)
H-ACUCCGCGUGGCCUCGGUCGUG-Lys-NH2;
(SEQ ID NO: 2)
H-AAGAAGCUAUUAAAAUCACAUGGGGA-Lys-NH2;
and
(SEQ ID NO: 3)
H-UAGGCAGCUCUCCCUAGCAUUGU-Lys-NH2.

8. The agent of claim 1, wherein the agent comprises 2′-deoxy 2′-fluoroarabino oligonucleotides.

9. The agent of claim 8, wherein the agent comprises an oligonucleotide sequence selected from the group consisting of:
(SEQ ID NO: 4)
ACUCCGCGUGGCCUCGGUCGUG;
(SEQ ID NO: 5)
AAGAAGCUAUUAAAAUCACAUGGGGA;
and
(SEQ ID NO: 6)
UAGGCAGCUCUCCCUAGCAUUGU.

10. The agent of claim 1, wherein the agent comprises LNA oligonucleotides.

11. The agent of claim 10, wherein the agent comprises an oligonucleotide sequence selected from the group consisting of:
(SEQ ID NO: 7)
ACUCCGCGUGGCCUCGGUCGUG;
(SEQ ID NO: 8)
AAGAAGCUAUUAAAAUCACAUGGGGA;
and
(SEQ ID NO: 9)
UAGGCAGCUCUCCCUAGCAUUGU.

12. The agent of claim 1, wherein the agent comprises a small molecule.

13. A method of treating an infection in a subject, comprising:
administering a therapeutically effective amount of the agent of claim 1 to the subject.

14. The method of claim 13, wherein the infection is SARS-CoV-2 infection.

15. A composition comprising the agent of claim 1.

16. A method of treating an infection in a subject, comprising:
administering a therapeutically effective amount of the composition of claim 15 to the subject.

17. The method of claim 16, wherein the infection is SARS-CoV-2 infection.

18. A method of treating or preventing the onset of a cytokine storm in a SARS-CoV-2 infection subject, comprising:
administering a therapeutically effective amount of the agent of claim 1 to the subject.

19. A method of treating or preventing the onset of a cytokine storm in a SARS-CoV-2 infection subject, comprising:
administering a therapeutically effective amount of the composition of claim 15 to the subject.

20. A method for treating SARS-CoV-2 infection, comprising:
targeting a SARS-CoV-2 s2m motif directly and/or its dimerization;
introducing small molecules or antisense molecules to the s2m motif; and
preventing and/or disrupting interactions of the s2m motif with miR-1307-3p, thereby releasing the miR-1307-3p to perform its normal cellular function;
or
targeting a SARS-CoV-2 3′-UTR Terminus directly; and
introducing small molecules or antisense molecules to the 3′ UTR Terminus motif preventing and/or disrupting interactions of the 3′ UTR Terminus with miR-760-3p, thereby releasing the miR-760-3p to perform its normal cellular function;
or
targeting a SARS-CoV-2 DIS-s2m extended directly; and
introducing small molecules or antisense molecules to the DIS-s2m extended motif preventing and/or disrupting interactions of the DIS-s2m extended with miR-34a-5p, thereby releasing the miR-34-5p to perform its normal cellular function.

21. A method for preventing the onset of a cytokine storm in a SARS-CoV-2 infection subject, comprising:
targeting a SARS-CoV-2 s2m motif directly and/or its dimerization;
introducing small molecules or antisense molecules to the s2m motif; and
preventing and/or disrupting interactions of the s2m motif with miR-1307-3p, thereby releasing the miR-1307-3p to perform its normal cellular function;
or
targeting a SARS-CoV-2 3′-UTR Terminus directly; and
introducing small molecules or antisense molecules to the 3′ UTR Terminus motif preventing and/or disrupting interactions of the 3′ UTR Terminus with miR-760-3p, thereby releasing the miR-760-3p to perform its normal cellular function;
or
targeting a SARS-CoV-2 DIS-s2m extended directly; and
introducing small molecules or antisense molecules to the DIS-s2m extended motif preventing and/or disrupting interactions of the DIS-s2m extended with miR-34a-5p, thereby releasing the miR-34-5p to perform its normal cellular function.
